(19) United States
(12) Patent Application Publication
(10) Pub. No.: US 2021/0002282 A1
(43) Pub. Date: Jan. 7, 2021

(54) SOLID FORMS OF IBRUTINIB

(71) Applicant: DR. REDDY'S LABORATORIES LIMITED, Hyderabad (IN)

(72) Inventors: Shanmukha Prasad GOPI, Visakhapatnam (IN); Venkata Narasayya SALADI, Hyderabad (IN); Vishweshwar PEDDY, Hyderabad (IN); Rajeev Budhdev REHANI, Hyderabad (IN)

(21) Appl. No.: 16/961,042

(22) PCT Filed: Jan. 9, 2019
(86) PCT No.: PCT/IB2019/050143

§ 371 Date: Jul. 9, 2020

(30) Foreign Application Priority Data
Jan. 9, 2018 (IN) 201841000955
Feb. 19, 2018 (IN) 201841006254
Apr. 2, 2018 (IN) 201841012446

(51) Int. Cl. C07D 487/04

(52) U.S. Cl. CPC C07D 487/04; C07B 2200/13

(57) ABSTRACT

The present invention describes solvates of Ibrutinib and processes for their preparation, pharmaceutical compositions comprising these solvate forms. The present invention also describes co-crystal, obtained from Ibrutinib and a neutral conformer, where both are solids at room temperature, process for their preparation, pharmaceutical compositions comprising the co-crystals and the use of these solids forms for the treatment of Burton's tyrosine kinase (BTK) mediated diseases.

### CROSS-REFERENCE TO RELATED APPLICATIONS

This application is a National Stage Application under 35 U.S.C. § 371 of PCT International Application No. PCT/M2019/050143, filed Jan. 9, 2019, which claims the benefit of Indian provisional patent application No. 201841000955 filed on 9 Jan. 2018; Indian provisional patent application No. 201841006254 filed on 19 Feb. 2018; Indian provisional patent application No. 201841012446 filed on 2 Apr. 2018, all of which are herein incorporated by reference in their entireties.

### FIELD OF THE INVENTION

The present application relates to solid forms of Ibrutinib, process for their preparation, and pharmaceutical compositions thereof.

### BACKGROUND OF THE INVENTION

The drug compound having the adopted name “Ibrutinib”, has a chemical name 1-{3R-3-(4-amino-3-(4-phenoxyphenyl)-1H-pyrazolo[3,4-d]pyrimidin-1-yl)piperidin-1-yl)}prop-2-en-1-one, and is represented by the structure of formula as below

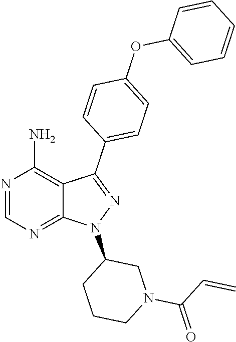 [SMILES: C=CC(=O)N1CCC[C@@H](n2nc(-c3ccc(Oc4ccccc4)cc3)c3c(N)ncnc32)C1]

Ibrutinib is an inhibitor of Bruton's tyrosine kinase (BTK) and is approved in US for the treatment of patients with mantle cell lymphoma and chronic lymphocytic leukemia who have received at least one prior therapy.

U.S. Pat. No. 7,514,444 discloses process for the preparation of Ibrutinib. The US '444 discloses isolation of Ibrutinib by flash chromatography using dichloromethane and methanol as eluents.

WO2013184572A1 application discloses crystalline, solvates and amorphous form of Ibrutinib. In particular, the application discloses polymorphic Forms A, B, C, D, E and F characterized by PXRD, IR, DSC and TGA. The WO '572 application discloses process for the preparation of amorphous form of Ibrutinib by dissolving Form A in dichloromethane. The solvent dichloromethane was removed under rotary evaporation to provide amorphous Ibrutinib. CN103694241A discloses crystal form A of Ibrutinib characterized by PXRD. CN103923084A discloses crystal forms II, III, IV, V, VI, VII and VIII of Ibrutinib, characterized by PXRD pattern. WO 2015145415A2 application discloses various solid forms of Ibrutinib designated as Form III, Form IV, Form V, Form VI, Form VII, Form VIII and Form IX. WO 2016022942A1 application discloses solid dispersions of ibrutinib. WO 2016025720A1 application discloses crystalline forms of Ibrutinib designated as Form G, Form J and Form K. WO2016139588A1 discloses crystalline forms of Ibrutinib designated as Form D1 to D13. WO2016079216A1 discloses solvates (Anisole, Chlorobenzene, DCM, 1,4-dioxane, Pyridine) of Ibrutinib. WO2016160598A1 discloses solvates (butyronitrile, 1,2-dimethoxy ethane, hexafluorobenzene, acetophenone, chlorobenzene, dimethylacetamide, benzyl acetate, or 1,1,2-trichloroethane) of Ibrutinib. WO2017029586A1 discloses crystalline forms of Ibrutinib designated as Form S1 to S4. EP3243824A1 discloses crystalline forms of Ibrutinib designated as Form a, (3, y, 6, c and WO2018000250A1 discloses crystalline form of Ibrutinib designated as Form III. US2018153895A1 discloses crystalline forms of Ibrutinib designated as APO-I as anhydrous form, methyl benzoate solvate (APO II) and methyl salicylate solvate (APO IV). Some other Chinese patent applications discloses crystalline forms and solvates of Ibrutinib.

WO2016160604A1 discloses co-crystal of Ibrutinib and a co-former with co-former being Benzoic acid, Succinic acid, 3-hydroxybenzoic acid, Nicotinamide, 4-aminobenzoic acid, salicylic acid, sorbic acid, fumaric acid, salicylamide, trans-cinnamic acid, 4-hydroxybenzoic acid, 1-hydroxys-naphthoic acid, sulfamic acid, 1,5-naphthalene disulfonic acid, 2-ethoxybenzamide, 4-aminosalicylic acid, or stearic acid.

WO2016156127A1 discloses co-crystal of Ibrutinib with carboxylic acid and the carboxylic acid being Benzoic acid, Fumaric acid, Succinic acid.

The existence and possible numbers of polymorphic forms for a given compound cannot be predicted, and there are no “standard” procedures that can be used to prepare polymorphic forms of a substance. This is well-known in the art, as reported, for example, by A. Goho, “Tricky Business,” Science News, Vol. 166(8), August 2004.

Despite of various crystalline forms of Ibrutinib, there remains a need for alternate solid forms of Ibrutinib and processes for preparing them.

The present invention describes solvates of Ibrutinib and processes for their preparation, pharmaceutical compositions comprising these solvate forms.

The present invention also describes co-crystal, obtained from Ibrutinib and a neutral conformer, where both are solids at room temperature.

The obtained solvates and co-crystals have a constant quality and have improved physicochemical properties, such as a higher solubility and dissolution rate, enhanced flow properties and enhanced stability.

### SUMMARY OF THE INVENTION

The present invention provides crystalline forms of Ibrutinib designated as Form D14, Form D15, Form D16, Form D17, Form D18, Form D19, Form D20a, Form D20, Form D21, Form D22, Form D23, Form D24, and processes for their preparation, pharmaceutical compositions comprising these forms, and their use for the treatment of Bruton's tyrosine kinase (BTK) mediated diseases.

### BRIEF DESCRIPTION OF THE DRAWINGS

FIG. 1 illustrates a characteristic PXRD pattern of crystalline Ibrutinib Form D14.

FIG. 2 illustrates a characteristic PXRD pattern of crystalline Ibrutinib Form D15.

FIG. 3 illustrates a characteristic PXRD pattern of co-crystal of Ibrutinib and Palmitic acid referred to as Form D16.

FIG. 4 illustrates a characteristic PXRD pattern of co-crystal of Ibrutinib and Decanoic acid referred to as Form D17.

FIG. 5 illustrates a characteristic PXRD pattern of crystalline Ibrutinib Form D18.

FIG. 6 illustrates a characteristic DSC thermogram of crystalline Ibrutinib Form D18.

FIG. 7 illustrates a characteristic TGA thermogram of crystalline Ibrutinib Form D18.

FIG. 8 illustrates a characteristic PXRD pattern of crystalline Ibrutinib Form D19.

FIG. 9 illustrates a characteristic DSC thermogram of crystalline Ibrutinib Form D19.

FIG. 10 illustrates a characteristic TGA thermogram of crystalline Ibrutinib Form D19.

FIG. 11 illustrates a characteristic PXRD pattern of crystalline Ibrutinib Form D20a.

FIG. 12 illustrates a characteristic PXRD pattern of crystalline Ibrutinib Form D20.

FIG. 13 illustrates a characteristic PXRD pattern of crystalline Ibrutinib Form D21.

FIG. 14 illustrates a characteristic PXRD pattern of crystalline Ibrutinib Form D22.

FIG. 15 illustrates a characteristic PXRD pattern of crystalline Ibrutinib Form D23.

FIG. 16 illustrates a characteristic PXRD pattern of crystalline Ibrutinib Form D24.

FIG. 17 ORTEP of Ibrutinib-Palmitic acid Co-crystal (Form-D16). Displacement ellipsoids were drawn at the 30% probability level for non-hydrogen atoms. Hydrogen atoms are shown as small spheres of arbitrary radii. Dashed line indicates hydrogen bonds.

### DETAILED DESCRIPTION

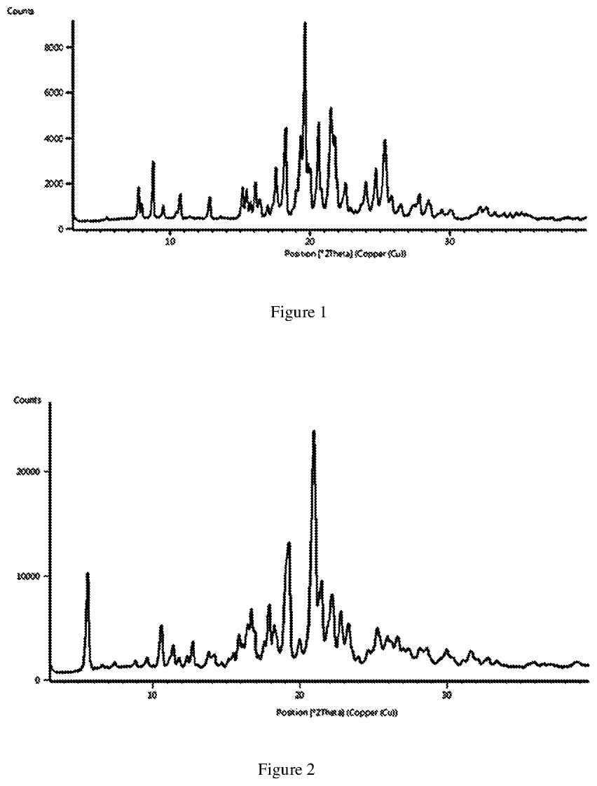

In an aspect, the present invention provides a crystalline form of Ibrutinib designated as Form D14. In an aspect, the crystalline form of Ibrutinib designated as Form D14 is benzyl alcohol solvate. In another aspect, the present invention provides a crystalline form of Ibrutinib designated as Form D14, characterized by X-ray powder diffraction pattern having peaks at about 7.71, 12.79 and 25.32±0.20 degrees 2-theta and also having peaks at about 9.46, 8.74, 19.60 and 24.66±0.20 degrees 2-theta. In another aspect, the present invention provides crystalline form of Ibrutinib designated as Form D14, characterized by an X-ray powder diffraction pattern as illustrated in FIG. 1.

In another aspect, the present invention provides a process for the preparation of crystalline form of Ibrutinib designated as Form D14, comprising the steps of:

a) mixing Ibrutinib and benzyl alcohol;

b) mixing an anti-solvent with content of step a); and

c) isolating the benzyl alcohol solvate of Ibrutinib.

The step a) may be performed at a temperature of about 10° C. to about the boiling point of the solvent. In a preferred embodiment, the step a) is performed at 20-80° C. In a more preferred embodiment, the step a) is performed at 20-40° C.

Step b) involves mixing with a suitable anti-solvent. The anti-solvent used is selected from methyl tert-butyl ether; aliphatic or alicyclic hydrocarbons such as hexane, heptane, pentane, cyclohexane, methyl cyclohexane, or the like. In an embodiment, the anti-solvent used is a mixture of methyl tert-butyl ether and n-heptane. The ratio of first anti-solvent to second anti-solvent in the mixture of anti-solvents may vary from about 1:1 to 1:10 by volume. Preferably the ratio is about 1:4 to 1:10 by volume.

In step b) the mixing of anti-solvent with contents of step a) may be performed at about 2-30° C. In a more preferred embodiment, the step b) is performed at 2-10° C.

Isolation in step c) may involve one or more methods including removal of solvent by techniques known in the art e.g. evaporation, distillation, filtration of precipitated solid and the like, cooling, concentrating the reaction mass, and the like. Stirring or other alternate methods such as shaking, agitation, and the like, may also be employed for the isolation. Isolation may be performed at a temperature of about 20-60° C. More preferably, the isolation is at temperature of about 20-40° C.

Drying may be done using any equipment such as a gravity oven, tray dryer, vacuum oven, Rotavapor®, air tray dryer, fluidized bed dryer, spin flash dryer, flash dryer, and the like. In an embodiment, the drying may be carried out at atmospheric pressure or under reduced pressure. In an embodiment, the drying may be carried out at a temperature of about 60° C., at a temperature of about 50° C., at a temperature of about 40° C. or at a temperature of about 30° C. The drying may be carried out for any time periods required for obtaining a desired quality, such as from about 15 minutes to several hours, or longer.

In another aspect, the present invention provides a crystalline form of Ibrutinib designated as Form D15. In an aspect, the crystalline form of Ibrutinib designated as Form D15 is benzyl alcohol solvate. In another aspect, the present invention provides a crystalline form of Ibrutinib designated as Form D15, characterized by X-ray powder diffraction pattern having peaks at about 9.58 and 25.23±0.20 degrees 2-theta and also having peaks at about 5.54, 11.33 and 17.86±0.20 degrees 2-theta. In another aspect, the present invention provides crystalline form of Ibrutinib designated as Form D15, characterized by an X-ray powder diffraction pattern as illustrated in FIG. 2.

In another aspect, the present invention provides a process for the preparation of crystalline form of Ibrutinib designated as Form D15, comprising the steps of:

a) mixing Ibrutinib and benzyl alcohol;

b) mixing an anti-solvent with content of step a); and

c) isolating the benzyl alcohol solvate of Ibrutinib.

The step a) may be performed at a temperature of about 10° C. to about the boiling point of the solvent. In a preferred embodiment, the step a) is performed at 20-80° C. In a more preferred embodiment, the step a) is performed at 20-40° C.

Step b) involves mixing with a suitable anti-solvent. The anti-solvent used is methyl tert-butyl ether, aliphatic or alicyclic hydrocarbons such as hexane, heptane, pentane, cyclohexane, methyl cyclohexane, or the like; or mixtures thereof. In an embodiment, the anti-solvent used is a mixture of methyl tert-butyl ether and n-heptane. The ratio of first anti-solvent to second anti-solvent in the mixture of anti-solvents may vary from about 1:1 to 1:10 by volume. Preferably the ratio is about 1:4 to 1:10 by volume.

In step b) the mixing of anti-solvent with contents of step a) may be performed at about 2-30° C. In a more preferred embodiment, the step b) is performed at 2-10° C.

Isolation in step c) may involve one or more methods including removal of solvent by techniques known in the art e.g. evaporation, distillation, filtration of precipitated solid and the like, cooling, concentrating the reaction mass, and the like. Stirring or other alternate methods such as shaking, agitation, and the like, may also be employed for the isolation. Isolation may be performed at a temperature of about 20-60° C. More preferably, the isolation is at temperature of about 20-40° C.

Drying may be done using any equipment such as a gravity oven, tray dryer, vacuum oven, Rotavapor®, air tray dryer, fluidized bed dryer, spin flash dryer, flash dryer, and the like. In an embodiment, the drying may be carried out at atmospheric pressure or under reduced pressure. In an embodiment, the drying may be carried out at a temperature of about 60° C., at a temperature of about 50° C., at a temperature of about 40° C. or at a temperature of about 30° C. The drying may be carried out for any time periods required for obtaining a desired quality, such as from about 15 minutes to several hours, or longer.

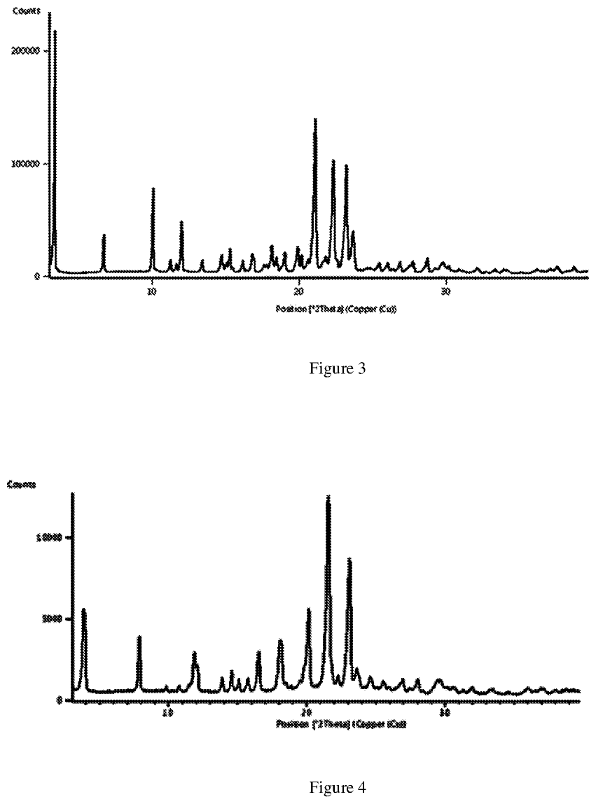

In an aspect, the present invention provides co-crystal of Ibrutinib and Palmitic acid. In another aspect, the present invention provides co-crystal of Ibrutinib and Palmitic acid, characterized by an x-ray powder diffraction pattern having peaks at about 3.35 and 6.69±0.20 degrees 2-theta, and also having peaks at about 10.03, 11.98, 13.38, 22.29 and 23.61±0.20 degrees 2-theta. In another aspect, the present invention provides co-crystal of Ibrutinib and Palmitic acid designated as Form D16, characterized by an X-ray powder diffraction pattern as illustrated in FIG. 3.

In another aspect, the present invention provides process for the preparation of co-crystal of Ibrutinib and Palmitic acid, comprising the steps of:

a) mixing Ibrutinib, Palmitic acid and a solvent or mixture of solvents;

b) obtaining a solution of the above contents;

c) crystallizing the solution to obtain the co-crystal.

The solvent or a mixture of two or more is/are selected from among aprotic solvents, methanol, ethanol, propanol, isopropanol, n-butanol, isobutanol, t-butanol, ethylene glycol, propylene glycol, acetone, methyl isobutyl ketone, methyl ethyl ketone, 1,4-dioxane, tetrahydrofuran, dimethylacetamide, dimethylformamide, dimethyl sulfoxide, acetonitrile or water. In an embodiment, the solvent is methanol.

Crystallization can be accomplished under cold conditions at a temperature of about 0-10° C., or by evaporating the solvent from the solution, the evaporation can be at 25-30° C. or at any other suitable temperature depending upon the solvent system, thereby obtaining the co-crystal.

The obtained co-crystal may optionally be dried and drying may be done using any equipment such as a gravity oven, tray dryer, vacuum oven, Rotavapor®, air tray dryer, fluidized bed dryer, spin flash dryer, flash dryer, and the like.

The drying may be carried out at atmospheric pressure or under reduced pressure. In an embodiment, the drying may be carried out at a temperature of about 60° C., at a temperature of about 50° C., at a temperature of about 40° C. or at a temperature of about 30° C. The drying may be carried out for any time periods required for obtaining a desired quality, such as from about 15 minutes to several hours, or longer.

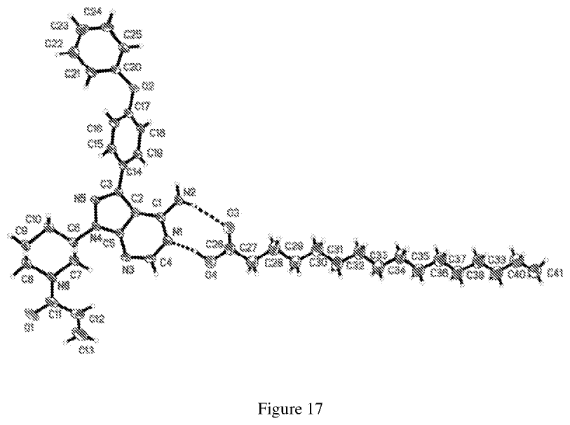

Single crystal X-ray structure of Ibrutinib and Palmitic acid reveals that the asymmetric unit consists of one molecule of Ibrutinib and one molecule of Palmitic acid. It is a 1:1 stoichiometric anhydrous co-crystal. Carboxylic acid functional group of Palmitic acid is forming robust ‘Carboxylic acid-Aminopyridine’ Supramolecular heterosynthon with amino-pyrimidine group of Ibrutinib through strong O—H . . . N and N—H . . . O hydrogen bonds as shown in FIG. 17. Such units are connected through (Amine) N—H . . . O═C (Amide) hydrogen bonds and forming a linear tape.

Crystal Data and Structure Refinement of Ibrutinib and Palmitic Acid 1:1 Co-Crystal.

| Empirical formula | C₂₅H₂₄N₆O₂, C₁₆ H₃₂ O₂ | |
|---|---|---|
| Formula weight | 696.91 | |
| Temperature | 294 | (2) K |
| Wavelength | 0.71073 | Å |
| Crystal system | Orthorhombic | |
| Space group | P2₁2₁2₁ | |
| Unit cell dimensions | a = 7.685 (2) Å, α = 90° | |
| | b = 9.764(3) Å, β = 90° | |
| | c = 52.921(16) Å, γ = 90° | |
| Volume | 3971 | (2) Å³ |
| Z | 4 | |
| Density | 1.166 | mg/m³ |
| Absorption coefficient | 0.076 | mm⁻¹ |
| F(000) | 1504 | |
| Crystal size | 0.20 × 0.18 × 0.10 mm³ | |
| Theta range for data collection | 2.223 to 24.995° | |
| Indexing ranges | −9 <= h <= 5, −10 <= | |
| | k <= 11, −62 <= l <= 62 | |
| Reflections collected | 21363 | |
| Independent reflections | 6975 [R(int) = 0.0593] | |
| Completeness to θ = 25° | 99.9% | |
| Refinement method | Full matrix least-squares on F² | |
| Data/restraints/parameters | 6975/0/473 | |
| Goodness of fit on F² | 1.041 | |
| Final R indices [I > 2θσ(I)] | R1 = 0.0599, wR2 = 0.1209 | |
| R indices (all data) | R1 = 0.1070, wR2 = 0.1385 | |
| Absolute structure parameter | −0.2 | (8) |
| Extinction coefficient | 0.0048 | (6) |

| | |
|---|---|
| Largest dif. Peak and hole | 0.254 and −0.164 e.Å⁻³ |
| Measurement | Bruker D8 QUEST |
| | PHOTON-100 Detector |
| Software used | SHELXTL-PLUS |

In an aspect, the present invention provides co-crystal of Ibrutinib and Decanoic acid. In an aspect, the present invention provides co-crystal of Ibrutinib and Decanoic acid in the 1:1 mole ratio.

In another aspect, the present invention provides co-crystal of Ibrutinib and Decanoic acid, characterized by an X-ray powder diffraction pattern having peaks at about 3.94 and 11.84±0.20 degrees 2-theta, and also having peaks at about 7.86, 14.55, 16.49, 18.04 and 24.59±0.20 degrees 2-theta.

In another aspect, the present invention provides co-crystal of Ibrutinib and Decanoic acid, characterized by an X-ray powder diffraction pattern as illustrated in FIG. 4.

In another aspect, the present invention provides process for the preparation of co-crystal of Ibrutinib and Decanoic acid, comprising the steps of:

a) mixing Ibrutinib, Decanoic acid and a solvent or mixture of solvents;

b) obtaining a solution of the above contents;

c) crystallizing the solution to obtain the co-crystal.

The solvent or a mixture of two or more is/are selected from among aprotic solvents, acetone, methyl isobutyl ketone, methyl ethyl ketone. In an embodiment, the solvent is acetone.

Crystallization can be accomplished under cold conditions at a temperature of about 0-10° C., or by evaporating the solvent from the solution, the evaporation can be at 25-30° C. or at any other suitable temperature depending upon the solvent system, thereby obtaining the co-crystal.

The obtained co-crystal may optionally be dried and drying may be done using any equipment such as a gravity oven, tray dryer, vacuum tray dryer, Rotavapor®, air tray dryer, fluidized bed dryer, spin flash dryer, flash dryer, and the like.

The drying may be carried out at atmospheric pressure or under reduced pressure. In an embodiment, the drying may be carried out at a temperature of about 60° C., at a temperature of about 50° C., at a temperature of about 40° C. or at a temperature of about 20° C. The drying may be carried out for any time periods required for obtaining a desired quality, such as from about 15 minutes to several hours, or longer.

In an aspect, the present invention provides a crystalline form of Ibrutinib designated as Form D18. In an aspect, the crystalline form of Ibrutinib designated as Form D18 is Octanoic acid solvate.

In another aspect, the present invention provides a crystalline form of Ibrutinib designated as Form D18, characterized by X-ray powder diffraction pattern having peaks at about 4.23, 8.43 and 12.65±0.20 degrees 2-theta and also having peaks at about 16.89, 17.33, 18.70, 21.13 and 24.05±0.20 degrees 2-theta.

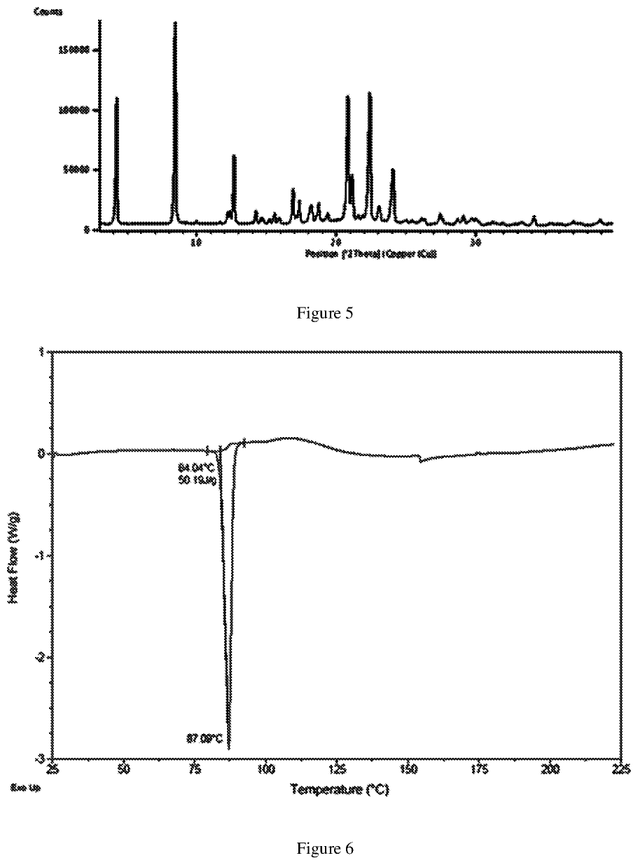

In another aspect, the present invention provides crystalline form of Ibrutinib designated as Form D18, characterized by an X-ray powder diffraction pattern as illustrated in FIG. 5.

The crystalline Form D18 has a differential scanning calorimetric thermogram substantially as shown in Figure. 6, which exhibits an endothermic peak at about 87.09° C.

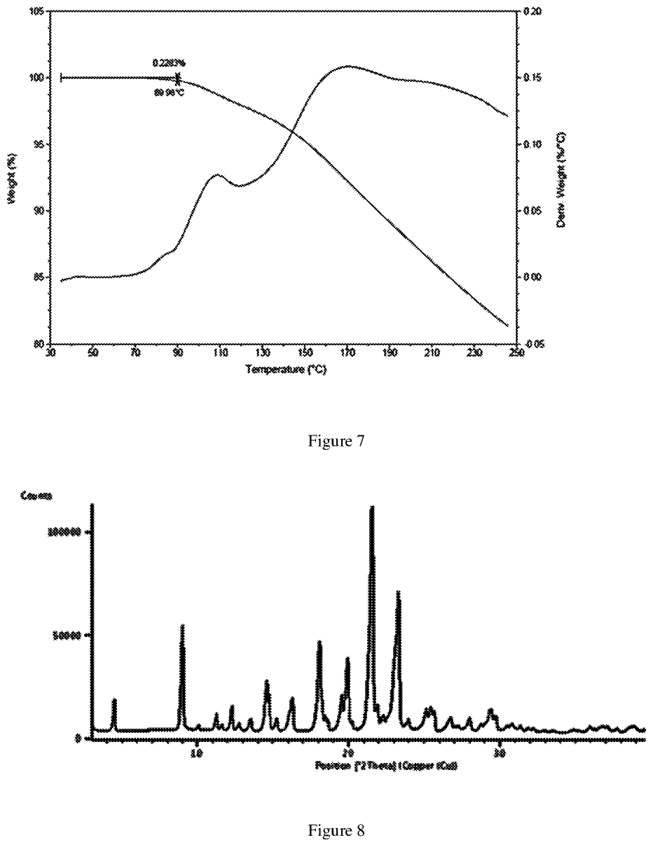

The crystalline Form D18 has a thermal gravimetric analysis thermogram substantially as shown in Figure. 7.

In another aspect, the present invention provides a process for the preparation of crystalline form of Ibrutinib designated as Form D18, comprising the steps of:

a) mixing Ibrutinib and Octanoic acid;

b) mixing an anti-solvent with content of step a); and

c) isolating the Octanoic acid solvate of Ibrutinib.

The step a) may be performed at a temperature of about 10° C. to about the boiling point of the solvent. In a preferred embodiment, the step a) is performed at 20-80° C. In a more preferred embodiment, the step a) is performed at 20-40° C.

Step b) involves mixing the solution obtained in step a) with a suitable anti-solvent. The anti-solvent used is selected from methyl tert-butyl ether, diisopropyl ether, pet ether or the like; aliphatic or alicyclic hydrocarbons such as hexane, heptane, pentane, cyclohexane, methyl cyclohexane, or the like; esters such as ethyl acetate, isopropyl acetate or the like. In an embodiment, the anti-solvent used is a mixture of methyl tert-butyl ether and n-heptane. In an embodiment, the anti-solvent used is ethyl acetate. The ratio of first anti-solvent to second anti-solvent in the mixture of anti-solvents may vary from about 1:1 to 1:10 by volume. Preferably the ratio is about 1:1 to 1:5 by volume.

In step b) the mixing of anti-solvent with contents of step a) may be performed at about 2-60° C. In a more preferred embodiment, the step b) is performed at about 20-50° C.

Isolation in step c) may involve one or more methods including removal of solvent by techniques known in the art e.g. evaporation, distillation, filtration of precipitated solid and the like, cooling, concentrating the reaction mass, and the like. Stirring or other alternate methods such as shaking, agitation, and the like, may also be employed for the isolation. Isolation may be performed at a temperature of about 2-60° C. More preferably, the isolation is at temperature of about 5-25° C.

Drying may be done using any equipment such as a gravity oven, tray dryer, vacuum oven, Rotavapor®, air tray dryer, fluidized bed dryer, spin flash dryer, flash dryer, and the like. In an embodiment, the drying may be carried out at atmospheric pressure or under reduced pressure. In an embodiment, the drying may be carried out at a temperature of about 60° C., at a temperature of about 50° C., at a temperature of about 40° C. or at a temperature of about 20° C. The drying may be carried out for any time periods required for obtaining a desired quality, such as from about 15 minutes to several hours, or longer.

In an aspect, the present invention provides a crystalline form of Ibrutinib designated as Form D19. In an aspect, the crystalline form of Ibrutinib designated as Form D19 is Hexanoic acid solvate.

In another aspect, the present invention provides a crystalline form of Ibrutinib designated as Form D19, characterized by X-ray powder diffraction pattern having peaks at about 4.48, 8.97 and 12.25±0.20 degrees 2-theta and also having peaks at about 18.05, 19.90, 21.49 and 23.26±0.20 degrees 2-theta.

In another aspect, the present invention provides crystalline form of Ibrutinib designated as Form D19, characterized by an X-ray powder diffraction pattern as illustrated in FIG. 8.

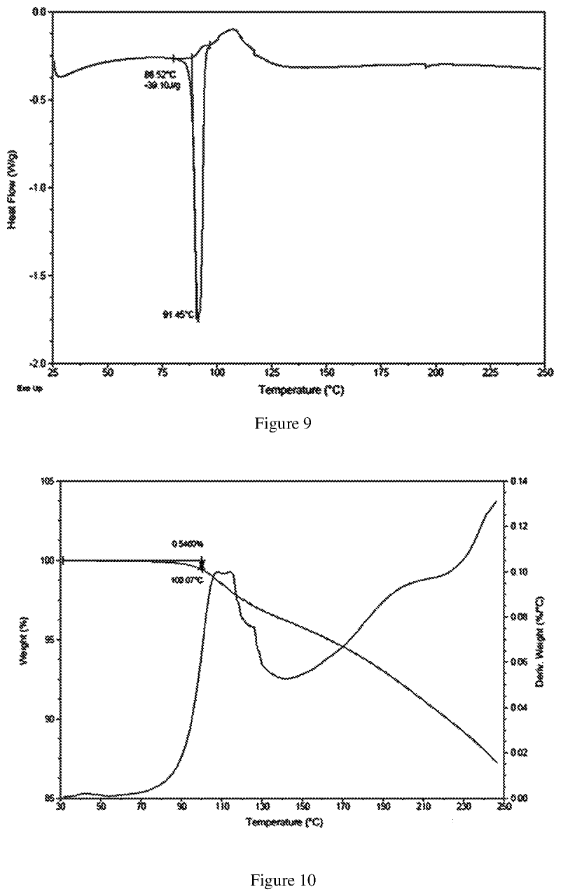

The crystalline Form D19 has a differential scanning calorimetric thermogram substantially as shown in Figure. 9, which exhibits an endothermic peak at about 91.45° C.

The crystalline Form D19 has a thermal gravimetric analysis thermogram substantially as shown in Figure. 10.

In another aspect, the present invention provides a process for the preparation of crystalline form of Ibrutinib designated as Form D19, comprising the steps of:

a) mixing Ibrutinib and Hexanoic acid;

b) mixing an anti-solvent with content of step a); and

c) isolating the Hexanoic acid solvate of Ibrutinib.

The step a) may be performed at a temperature of about 10° C. to about the boiling point of the solvent. In a preferred embodiment, the step a) is performed at 2-60° C. In a more preferred embodiment, the step a) is performed at 20-50° C.

Step b) involves mixing with a suitable anti-solvent. The anti-solvent used is methyl tert-butyl ether, diisopropyl ether, pet ether or the like; aliphatic or alicyclic hydrocarbons such as hexane, heptane, pentane, cyclohexane, methyl cyclohexane, or the like; or mixtures thereof. In an embodiment, the anti-solvent used is a mixture of methyl tert-butyl ether and n-heptane. In another embodiment, the anti-solvent used is a mixture of methyl tert-butyl ether and n-hexane. The ratio of first anti-solvent to second anti-solvent in the mixture of anti-solvents may vary from about 1:1 to 1:20 by volume.

In step b) the mixing of anti-solvent with contents of step a) may be performed at about 2-60° C. In a more preferred embodiment, the step b) is performed at 20-50° C.

Isolation in step c) may involve one or more methods including removal of solvent by techniques known in the art e.g. evaporation, distillation, filtration of precipitated solid and the like, cooling, concentrating the reaction mass, and the like. Stirring or other alternate methods such as shaking, agitation, and the like, may also be employed for the isolation. Isolation may be performed at a temperature of about 0-60° C. More preferably, the isolation is at temperature of about 0-40° C.

Drying may be done using any equipment such as a gravity oven, tray dryer, vacuum oven, Rotavapor®, air tray dryer, fluidized bed dryer, spin flash dryer, flash dryer, and the like. In an embodiment, the drying may be carried out at atmospheric pressure or under reduced pressure. In an embodiment, the drying may be carried out at a temperature of about 60° C., at a temperature of about 50° C., at a temperature of about 40° C. or at a temperature of about 20° C. The drying may be carried out for any time periods required for obtaining a desired quality, such as from about 15 minutes to several hours, or longer.

Both the Form D18 (Octanoic acid solvate) and Form D19 (Hexanoic acid solvate) of the present applications are non-hygroscopic, chemically and physically stable and suitable for pharmaceutical formulation. The following tables provides the stability data of both Form D18 and Form D19.

Stability data of Form D18 (Octanoic acid solvate)

| Storage | Time | | % Water | % Total | Octanoic acid | |
|---|---|---|---|---|---|---|
| condition | Period | Description | content | Impurities | content (%) | PXRD |
| 40° C. ± 2° C. | Initial | White | 0.13 | 0.38 | 25.24 | Crystalline |
| 75% RH ± 5RH | | colored solid | | | | |
| | 1 Month | White | 0.16 | 0.45 | 24.70 | Matches with |
| | | colored solid | | | | Initial |
| | 3 Months | White | 0.29 | 0.45 | 24.99 | Matches with |
| | | colored solid | | | | Initial |
| 25° C. ± 2° C. | Initial | White | 0.13 | 0.38 | 25.24 | Crystalline |
| 60% RH ± 5RH | | colored solid | | | | |
| | 1 Month | White | 0.10 | 0.46 | 24.70 | Matches with |
| | | colored solid | | | | Initial |
| | 3 Months | White | 0.19 | 0.39 | 25.57 | Matches with |
| | | colored solid | | | | Initial |

Stability data of Form D19 (Hexanoic acid acid solvate)

| Storage | Time | | % Water | % Total | Hexanoic acid | |
|---|---|---|---|---|---|---|
| condition | Period | Description | content | Impurities | content (%) | PXRD |
| 40° C. ± 2° C. | Initial | Off-White | 0.19 | 0.41 | 23.13 | Crystalline |
| 75% RH ± 5RH | | colored solid | | | | |
| | 1 Month | Off-White | 0.13 | 0.49 | 21.48 | Matches with |
| | | colored solid | | | | Initial |
| | 3 Months | Off-White | 0.11 | 0.50 | 20.79 | Matches with |
| | | coloured solid | | | | Initial |
| 25° C. ± 2° C. | Initial | Off-White | 0.19 | 0.41 | 23.13 | Crystalline |
| 60% RH ± 5RH | | colored solid | | | | |
| | 1 Month | Off-White | 0.20 | 0.42 | 21.73 | Matches with |
| | | colored solid | | | | Initial |
| | 3 Months | Off-White | 0.09 | 0.42 | 21.24 | Matches with |
| | | colored solid | | | | Initial |

Form D18 and Form D19 can be micronized to get the desired particle size suitable for pharmaceutical formulation.

In an aspect, the present invention provides a crystalline form of Ibrutinib designated as Form D20a. In an aspect, the crystalline form of Ibrutinib designated as Form D20a is Butyric acid solvate.

In another aspect, the present invention provides a crystalline form of Ibrutinib designated as Form D20a, characterized by X-ray powder diffraction pattern having peaks at about 4.69, 9.38, 11.39, 14.90 and 16.82±0.20 degrees 2-theta and also having peaks at about 18.07, 20.43, 21.06, 22.23 and 24.11±0.20 degrees 2-theta.

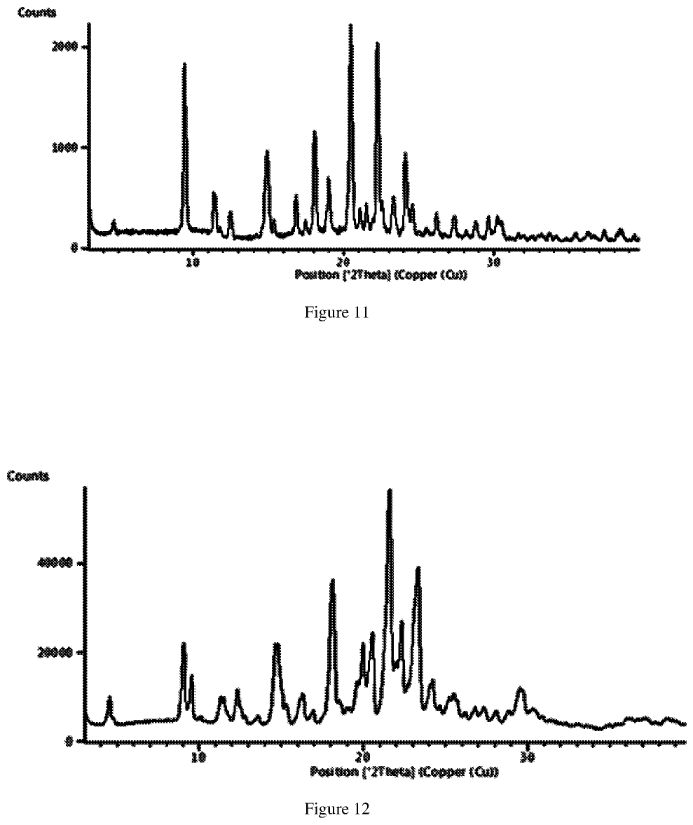

In another aspect, the present invention provides crystalline form of Ibrutinib designated as Form D20a, characterized by an X-ray powder diffraction pattern as illustrated in FIG. 11.

In another aspect, the present invention provides a process for the preparation of crystalline form of Ibrutinib designated as Form D20a, comprising the steps of:

a) mixing Ibrutinib and Butyric acid;

b) mixing an anti-solvent with content of step a); and

c) isolating the Butyric acid solvate of Ibrutinib.

Step a) may be performed at a temperature of about 10° C. to about the boiling point of the solvent. In a preferred embodiment, the step a) is performed at 20-80° C. In a more preferred embodiment, the step a) is performed at 20-40° C.

Step b) involves mixing with a suitable anti-solvent. The anti-solvent used is selected from methyl tert-butyl ether, diisopropyl ether, pet ether or the like; aliphatic or alicyclic hydrocarbons such as hexane, heptane, pentane, cyclohexane, methyl cyclohexane, or the like; esters such as ethyl acetate, isopropyl acetate or the like. In an embodiment, the anti-solvent used is a mixture of methyl tert-butyl ether and n-heptane. In an embodiment, the anti-solvent used is ethyl acetate. The ratio of first anti-solvent to second anti-solvent in the mixture of anti-solvents may vary from about 1:1 to 1:10 by volume. Preferably the ratio is about 1:1 to 1:5 by volume.

In step b) the mixing of anti-solvent with contents of step a) may be performed at about 2-60° C. In a more preferred embodiment, the step b) is performed at about 20-50° C.

Isolation in step c) may involve one or more methods including removal of solvent by techniques known in the art e.g. evaporation, distillation, filtration of precipitated solid and the like, cooling, concentrating the reaction mass, and the like. Stirring or other alternate methods such as shaking, agitation, and the like, may also be employed for the isolation. Isolation may be performed at a temperature of about 2-60° C. More preferably, the isolation is at temperature of about 5-25° C.

In another aspect, Form D20a is dried to obtain Form D20.

In an aspect, the crystalline form of Ibrutinib designated as Form D20 is Butyric acid solvate. In another aspect, the present invention provides a crystalline form of Ibrutinib designated as Form D20, characterized by X-ray powder diffraction pattern having peaks at about 4.52, 9.03, 9.52 and 12.32±0.20 degrees 2-theta and also having peaks at about 18.05, 19.96, 20.49 and 21.55±0.20 degrees 2-theta.

In another aspect, the present invention provides crystalline form of Ibrutinib designated as Form D20, characterized by an X-ray powder diffraction pattern as illustrated in FIG. 12.

Drying may be done using any equipment such as a gravity oven, tray dryer, vacuum oven, Rotavapor®, air tray dryer, fluidized bed dryer, spin flash dryer, flash dryer, and the like. In an embodiment, the drying may be carried out at atmospheric pressure or under reduced pressure. In an embodiment, the drying may be carried out at a temperature of about 60° C., at a temperature of about 50° C., at a temperature of about 40° C. or at a temperature of about 30° C. The drying may be carried out for any time periods required for obtaining a desired quality, such as from about 15 minutes to several hours, or longer. In an embodiment, Form D20a is dried at 25° C. in vacuum oven for about 12-72 hours to obtain Form D20.

In an aspect, the present invention provides a crystalline form of Ibrutinib designated as Form D21. In an aspect, the crystalline form of Ibrutinib designated as Form D21 is Pentanoic acid solvate.

In another aspect, the present invention provides a crystalline form of Ibrutinib designated as Form D21, characterized by X-ray powder diffraction pattern having peaks at about 6.80, 10.57, 13.60 and 16.95±0.20 degrees 2-theta and also having peaks at about 17.70, 19.86, 21.24, 21.89, 22.87, 23.63 and 25.11±0.20 degrees 2-theta.

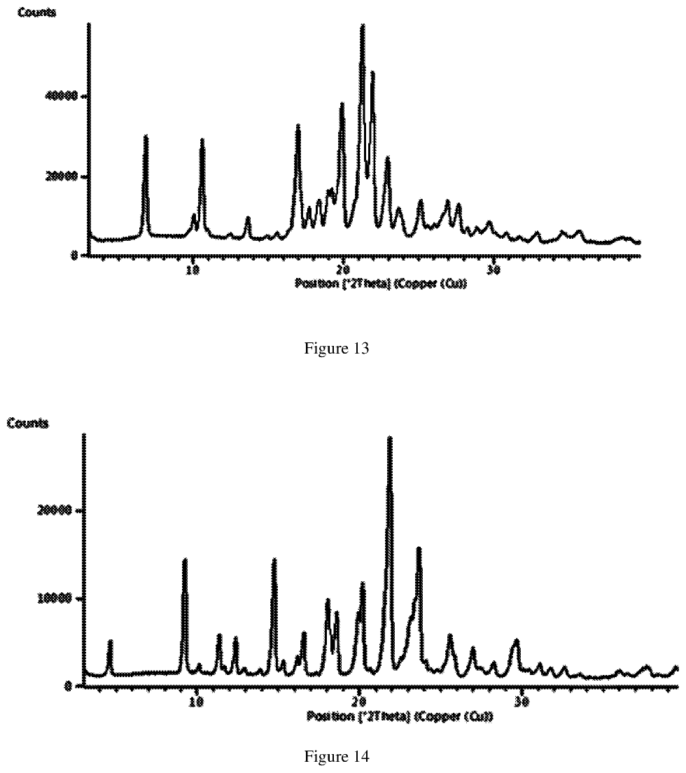

In another aspect, the present invention provides crystalline form of Ibrutinib designated as Form D21, characterized by an X-ray powder diffraction pattern as illustrated in FIG. 13.

In another aspect, the present invention provides a process for the preparation of crystalline form of Ibrutinib designated as Form D21, comprising the steps of:

a) mixing Ibrutinib and Pentanoic acid;

b) mixing an anti-solvent with content of step a); and

c) isolating the Pentanoic acid solvate of Ibrutinib.

The step a) may be performed at a temperature of about 20° C. to about the boiling point of the solvent. In a preferred embodiment, the step a) is performed at 20-80° C. In a more preferred embodiment, the step a) is performed at 20-40° C.

Step b) involves mixing with a suitable anti-solvent. The anti-solvent used is selected from methyl tert-butyl ether, diisopropyl ether, pet ether or the like; aliphatic or alicyclic hydrocarbons such as hexane, heptane, pentane, cyclohexane, methyl cyclohexane, or the like; esters such as ethyl acetate, isopropyl acetate or the like. In an embodiment, the anti-solvent used is a mixture of methyl tert-butyl ether and n-heptane. In an embodiment, the anti-solvent used is ethyl acetate. The ratio of first anti-solvent to second anti-solvent in the mixture of anti-solvents may vary from about 1:1 to 1:10 by volume. Preferably the ratio is about 1:1 to 1:5 by volume.

In step b) the mixing of anti-solvent with contents of step a) may be performed at about 2-60° C. In a more preferred embodiment, the step b) is performed at about 20-50° C.

Isolation in step c) may involve one or more methods including removal of solvent by techniques known in the art e.g. evaporation, distillation, filtration of precipitated solid and the like, cooling, concentrating the reaction mass, and the like. Stirring or other alternate methods such as shaking, agitation, and the like, may also be employed for the isolation. Isolation may be performed at a temperature of about 20-50° C. More preferably, the isolation is at temperature of about 20-30° C.

Drying may be done using any equipment such as a gravity oven, tray dryer, vacuum oven, Rotavapor®, air tray dryer, fluidized bed dryer, spin flash dryer, flash dryer, and the like. In an embodiment, the drying may be carried out at atmospheric pressure or under reduced pressure. In an embodiment, the drying may be carried out at a temperature of about 60° C., at a temperature of about 50° C., at a temperature of about 40° C. or at a temperature of about 20° C. The drying may be carried out for any time periods required for obtaining a desired quality, such as from about 15 minutes to several hours, or longer.

In an aspect, the present invention provides a crystalline form of Ibrutinib designated as Form D22. In an aspect, the crystalline form of Ibrutinib designated as Form D22 is Pentanoic acid solvate.

In another aspect, the present invention provides a crystalline form of Ibrutinib designated as Form D22, characterized by X-ray powder diffraction pattern having peaks at about 4.61, 9.23, 11.34, 12.33 and 14.71±0.20 degrees 2-theta and also having peaks at about 18.03, 18.56, 20.17, 21.83 and 25.57±0.20 degrees 2-theta.

In another aspect, the present invention provides crystalline form of Ibrutinib designated as Form D22, characterized by an X-ray powder diffraction pattern as illustrated in FIG. 14.

In another aspect, the present invention provides a process for the preparation of crystalline form of Ibrutinib designated as Form D22, comprising the steps of:

a) mixing Ibrutinib and Pentanoic acid;

b) mixing an anti-solvent with content of step a); and

c) isolating the Pentanoic acid solvate of Ibrutinib.

Step a) may be performed at a temperature of about 20° C. to about the boiling point of the solvent. In a preferred embodiment, the step a) is performed at 20-80° C. In a more preferred embodiment, the step a) is performed at 20-40° C.

Step b) involves mixing with a suitable anti-solvent. The anti-solvent used is selected from methyl tert-butyl ether, diisopropyl ether, pet ether or the like; aliphatic or alicyclic hydrocarbons such as hexane, heptane, pentane, cyclohexane, methyl cyclohexane, or the like; esters such as ethyl acetate, isopropyl acetate or the like. In an embodiment, the anti-solvent used is a mixture of methyl tert-butyl ether and n-heptane. In an embodiment, the anti-solvent used is ethyl acetate. The ratio of first anti-solvent to second anti-solvent in the mixture of anti-solvents may vary from about 1:1 to 1:10 by volume. Preferably the ratio is about 1:1 to 1:5 by volume.

In step b) the mixing of anti-solvent with contents of step a) may be performed at about 2-8° C. In a more preferred embodiment, the step b) is performed at about 3-6° C.

Isolation in step c) may involve one or more methods including removal of solvent by techniques known in the art e.g. evaporation, distillation, filtration of precipitated solid and the like, cooling, concentrating the reaction mass, and the like. Stirring or other alternate methods such as shaking, agitation, and the like, may also be employed for the isolation. Isolation may be performed at a temperature of about 2-25° C. More preferably, the isolation is at temperature of about 5-20° C.

Drying may be done using any equipment such as a gravity oven, tray dryer, vacuum oven, Rotavapor®, air tray dryer, fluidized bed dryer, spin flash dryer, flash dryer, and the like. In an embodiment, the drying may be carried out at atmospheric pressure or under reduced pressure. In an embodiment, the drying may be carried out at a temperature of about 60° C., at a temperature of about 50° C., at a temperature of about 40° C. or at a temperature of about 20° C. The drying may be carried out for any time periods required for obtaining a desired quality, such as from about 15 minutes to several hours, or longer.

In an embodiment, the invention provides solvates and/or co-crystals of Ibrutinib with C4-C16 carboxylic acid.

In an aspect, the present invention provides a crystalline form of Ibrutinib designated as Form D23. In an aspect, the crystalline form of Ibrutinib designated as Form D23 is nonanoic acid solvate.

In another aspect, the present invention provides a crystalline form of Ibrutinib designated as Form D23, characterized by X-ray powder diffraction pattern having peaks at about 4.1, 8.14 and 12.18±0.20 degrees 2-theta and also having peaks at about 16.81, 21.89 and 23.48±0.20 degrees 2-theta.

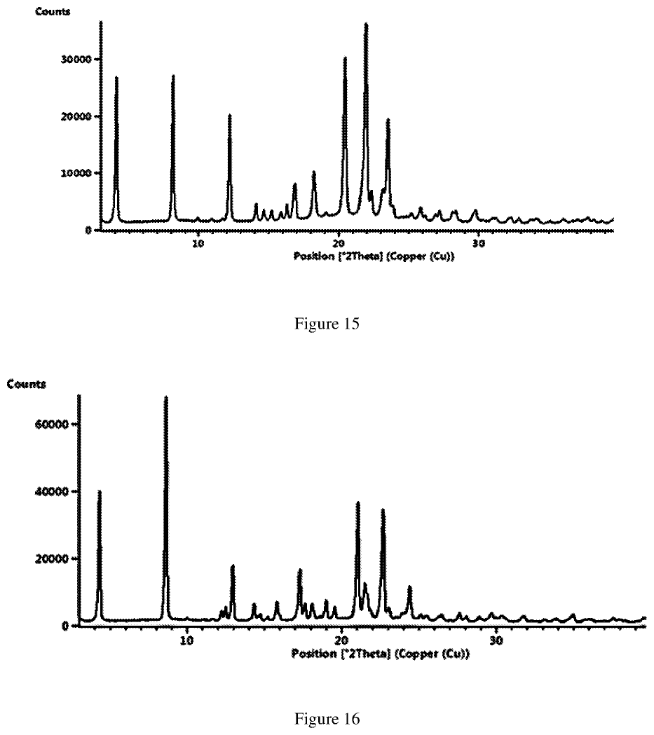

In another aspect, the present invention provides crystalline form of Ibrutinib designated as Form D23, characterized by an X-ray powder diffraction pattern as illustrated in FIG. 15.

In another aspect, the present invention provides a process for the preparation of crystalline form of Ibrutinib designated as Form D23, comprising the steps of:

a) mixing Ibrutinib and Nonanoic acid;

b) mixing anti-solvent with content of step a); and

c) isolating the Nonanoic acid solvate of Ibrutinib.

Step a) may be performed at a temperature of about 10° C. to about the boiling point of the solvent. In a preferred embodiment, the step a) is performed at 20-80° C. In a more preferred embodiment, the step a) is performed at 20-40° C.

Step b) involves mixing with a suitable anti-solvent. The anti-solvent used is selected from methyl tert-butyl ether, diisopropyl ether, pet ether or the like; aliphatic or alicyclic hydrocarbons such as hexane, heptane, pentane, cyclohexane, methyl cyclohexane, or the like; esters such as ethyl acetate, isopropyl acetate or the like. In an embodiment, the anti-solvent used is a mixture of methyl tert-butyl ether and n-heptane. In an embodiment, the anti-solvent used is ethyl acetate. The ratio of first anti-solvent to second anti-solvent in the mixture of anti-solvents may vary from about 1:1 to 1:10 by volume. Preferably the ratio is about 1:1 to 1:7 by volume.

In step b) the mixing of anti-solvent with contents of step a) may be performed at about 20-60° C. In a more preferred embodiment, the step b) is performed at about 20-50° C.

Isolation in step c) may involve one or more methods including removal of solvent by techniques known in the art e.g. evaporation, distillation, filtration of precipitated solid and the like, cooling, concentrating the reaction mass, and the like. Stirring or other alternate methods such as shaking, agitation, and the like, may also be employed for the isolation. Isolation may be performed at a temperature of about 0-50° C. More preferably, the isolation is at temperature of about 0-40° C.

Drying may be done using any equipment such as a gravity oven, tray dryer, vacuum oven, Rotavapor®, air tray dryer, fluidized bed dryer, spin flash dryer, flash dryer, and the like. In an embodiment, the drying may be carried out at atmospheric pressure or under reduced pressure. In an embodiment, the drying may be carried out at a temperature of about 60° C., at a temperature of about 50° C., at a temperature of about 40° C. or at a temperature of about 20° C. The drying may be carried out for any time periods required for obtaining a desired quality, such as from about 15 minutes to several hours, or longer.

In an aspect, the present invention provides a crystalline form of Ibrutinib designated as Form D24. In an aspect, the crystalline form of Ibrutinib designated as Form D24 is Heptanoic acid solvate.

In another aspect, the present invention provides a crystalline form of Ibrutinib designated as Form D24, characterized by X-ray powder diffraction pattern having peaks at about 4.31, 8.61 and 12.92±0.20 degrees 2-theta and also having peaks at about 14.32, 17.26, 21.02, 22.64 and 24.37±0.20 degrees 2-theta.

In another aspect, the present invention provides crystalline form of Ibrutinib designated as Form D24, characterized by an X-ray powder diffraction pattern as illustrated in FIG. 16.

In another aspect, the present invention provides a process for the preparation of crystalline form of Ibrutinib designated as Form D24, comprising the steps of:

a) mixing Ibrutinib and Heptanoic acid;

b) mixing anti-solvent with content of step a); and

c) isolating the Heptanoic acid solvate of Ibrutinib.

Step a) may be performed at a temperature of about 10° C. to about the boiling point of the solvent. In a preferred embodiment, the step a) is performed at 10-60° C. In a more preferred embodiment, the step a) is performed at 10-50° C.

Step b) involves mixing with a suitable anti-solvent. The anti-solvent used is methyl tert-butyl ether, diisopropyl ether, pet ether or the like; aliphatic or alicyclic hydrocarbons such as hexane, heptane, pentane, cyclohexane, methyl cyclohexane, or the like; water; or mixtures thereof. In an embodiment, the anti-solvent used is a mixture of methyl tert-butyl ether and n-pentane. In another embodiment, the anti-solvent used is a mixture of water and n-hexane. The ratio of first anti-solvent to second anti-solvent in the mixture of anti-solvents may vary from about 1:1 to 1:10 by volume.

In step b) the mixing of anti-solvent with contents of step a) may be performed at about 10-60° C. In a more preferred embodiment, the step b) is performed at 10-50° C.

Isolation in step c) may involve one or more methods including removal of solvent by techniques known in the art e.g. evaporation, distillation, filtration of precipitated solid and the like, cooling, concentrating the reaction mass, and the like. Stirring or other alternate methods such as shaking, agitation, and the like, may also be employed for the isolation. Isolation may be performed at a temperature of about 0-60° C. More preferably, the isolation is at temperature of about 0-40° C.

Drying may be done using any equipment such as a gravity oven, tray dryer, vacuum oven, Rotavapor®, air tray dryer, fluidized bed dryer, spin flash dryer, flash dryer, and the like. In an embodiment, the drying may be carried out at atmospheric pressure or under reduced pressure. In an embodiment, the drying may be carried out at a temperature of about 60° C., at a temperature of about 50° C., at a temperature of about 40° C. or at a temperature of about 20° C. The drying may be carried out for any time periods required for obtaining a desired quality, such as from about 15 minutes to several hours, or longer.

In an embodiment, the invention provides solvates and/or co-crystals of Ibrutinib with C4-C16 carboxylic acid.

Any particular form of Ibrutinib may be used as input material for preparing Ibrutinib Form D14, D15, D16, D17, D18, D19, D20a, D20, D21, D22, D23 and D24 as described in this patent application.

In another aspect, the present invention provides pharmaceutical composition comprising a therapeutically effective amount of crystalline form of Ibrutinib Form D14, D15, D16, D17, D18, D19, D20a, D20, D21, D22, D23 and D24 as mentioned above along with one or more suitable pharmaceutically acceptable carriers/excipients.

Further, the pharmaceutical composition of the invention may be any pharmaceutical form which contains crystalline forms of the invention. The pharmaceutical composition may be solid form such as tablets, powders, capsule, liquid suspension or an injectable composition along with any suitable carrier well known in the prior art. The dosage forms can also be prepared as sustained, controlled, modified and immediate release dosage forms. Suitable excipients and the amounts to use may be radially determined by the standard procedures and reference works in the field, e.g. the buffering agents, sweetening agents, binders, diluents, fillers, lubricants, wetting agents, disintegrates etc.

All PXRD data reported herein are obtained using a PANalytical X-ray Diffractometer, with copper Kα radiation.

### Instrumental Parameters.

Model & Detector

| | |
|---|---|
| Model | PANalytical & |
| | X'Pert PRO |
| Detector | X'Celerator |

Instrument Setting

| Goniometer | Theta/Theta | |
|---|---|---|
| Mode of Collection | Reflection | |
| Measuring circle | 240 | mm |

| | |
|---|---|
| Radiation | Cu K-alpha (Wave |
| | length = 1.5406 Å) |

Scan Parameters

| Voltage (kV) and | 45 kV and 40 mA | |
|---|---|---|
| Current (mA) | | |
| Scan range (°2θ) | 3-40 | |
| Step size (°2θ) | 0.017 | |
| Scan Step Time (s) | 64.77 | |
| Run Time (min) | 20 | min |
| Scan mode | Continuous | |
| Divergent slit (°) | 0.5 | |
| Anti-scattering slit (mm) | 5.5 | mm |

| | |
|---|---|
| Rotation/min | On |

### Differential Scanning Calorimetry (DSC)

### Analytical Instrument: TA Instruments Discovery (DSC)

Heating rate: 10° C. per minute.
Purge gas: nitrogen

### Thermal Gravimetric Analysis (TGA)

### Analytical Instrument: TA Instruments Q500 (TGA)

Heating rate: 10° C. per minute.
Purge gas: nitrogen.

### Definitions

The following definitions are used in connection with the present application unless the context indicates otherwise.

The terms “about,” the like are to be construed as modifying a term or value such that it is not an absolute. Such terms will be defined by the circumstances and the terms that they modify as those terms are understood by those of skill in the art. This includes, at very least, the degree of expected experimental error, technique error and instrument error for a given technique used to measure a value. The term “about” when used in the present application preceding a number and referring to it, is meant to designate any value which lies within the range of ±10%, preferably within a range of ±5%, more preferably within a range of ±2%, still more preferably within a range of ±1% of its value. For example “about 10” should be construed as meaning within the range of 9 to 11, preferably within the range of 9.5 to 10.5, more preferably within the range of 9.8 to 10.2, and still more preferably within the range of 9.9 to 10.1.

All percentages and ratios used herein are by weight of the total composition and all measurements made are at about 25° C. and about atmospheric pressure, unless otherwise designated. All temperatures are in degrees Celsius unless specified otherwise. As used herein, the terms “comprising” and “comprises” mean the elements recited, or their equivalents in structure or function, plus any other element or elements which are not recited. The terms “having” and “including” are also to be construed as open ended. All ranges recited herein include the endpoints, including those that recite a range between two values. Whether so indicated or not, all values recited herein are approximate as defined by the circumstances, including the degree of expected experimental error, technique error, and instrument error for a given technique used to measure a value.

The term “optional” or “optionally” is taken to mean that the event or circumstance described in the specification may or may not occur, and that the description includes instances where the event occurs and instances where it does not.

The term “anti-solvent” may be taken to mean a solvent in which Ibrutinib have low solubility.

“Co-crystal” is a crystal formed by two or more non-identical molecules, in which the starting components are solid at room conditions when they are in their pure form, and wherein the two or more co-crystal components form aggregates that are characterized by being linked by intermolecular interactions—such as the Van der Waals forces, π-stacking, hydrogen bonding or electrostatic interactions—but without forming covalent bonds.

Certain specific aspects and embodiments of the present application will be explained in greater detail with reference to the following examples, which are provided only for purposes of illustration and should not be construed as limiting the scope of the application in any manner. Reasonable variations of the described procedures are intended to be within the scope of the present invention. While particular aspects of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

### EXAMPLES

### Example 1: Preparation of Crystalline Ibrutinib Form D14

10.0 g of ibrutinib and 40 mL of benzyl alcohol were taken together and stirred at 25° C. The contents were cooled to 3° C. and 240 mL of n-heptane and 160 mL of methyl tert-butyl ether were added and stirred for 22-23 hours at 3° C. The contents were filtered at 25° C. and dried in vacuum tray drier at 40° C. for 18-20 hours to afford the title compound.

Result: Form D14; PXRD pattern: FIG. 1

### Example 2: Preparation of Crystalline Ibrutinib Form D15

2.5 g of ibrutinib and 10 mL of benzyl alcohol were taken together and stirred at 25° C. The contents were heated to 50° C. and then cooled to 5° C. To the cooled contents were added 60 mL of n-heptane and 40 mL of methyl tert-butyl ether and stirred for 17-18 hours at 5° C. The contents were filtered at 25° C. and dried in vacuum tray drier at 40° C. to afford the title compound.

Result: Form D15; PXRD pattern: FIG. 2

### Example 3: Preparation of Co-Crystal of Ibrutinib and Palmitic Acid (Form D16)

4.14 g of Ibrutinib, 2.56 g of Palmitic acid and 200 mL of methanol was taken together and stirred at 25° C. The contents were heated to 50° C. for one hour and cooled to 25° C. The contents were stirred at 25° C. for two hours and filtered using 0.45μ syringe filter. The filtrate was dried in open atmosphere at 25° C. for about 3 days to afford the title compound.

Result: Form D16. PXRD pattern: FIG. 3

### Example 4: Preparation of Co-Crystal of Ibrutinib and Decanoic Acid (Form D17)

4.41 g of Ibrutinib, 2.73 g of Decanoic acid and 80 mL of acetone was taken together and stirred at 25° C. The contents were heated to 40° C. and then cooled to 25° C. The contents were taken in a beaker and covered with pin holed aluminum foil. The solvent was allowed to evaporate for 6 days at 25° C. to afford solid compound. The isolated solid was dried in vacuum tray drier at 25° C. for about 30 hours followed by drying at 40° C. for 24 hours to afford the title compound 18-20 hours to afford the title compound.

Result: Form D17.

### Example 5: Preparation of Crystalline Ibrutinib Form D18

32.0 g of Ibrutinib and 150 mL of Octanoic acid were taken together and stirred at 25° C. The contents were heated to 50° C. and then cooled to 25° C. 200 mL of n-heptane and 150 mL of methyl tert-butyl ether were added and stirred for 1-2 hours at 5° C. The contents were filtered at 25° C. and dried in vacuum tray drier at 40° C. for 24-48 hours to afford the title compound.

Result: Form D18

### Example 6: Preparation of Crystalline Ibrutinib Form D18

0.5 g of Ibrutinib and 3 mL of Octanoic acid were taken together and stirred at 25° C. The contents were heated to 50° C. and then cooled to 25° C. and 3 mL of ethyl acetate was added. The contents were filtered using 0.45μ syringe filter. The filtrate was dried in open atmosphere at 25° C. for about 3 days to afford the title compound.

Result: Form D18

### Example 7: Preparation of Crystalline Ibrutinib Form D18

750 g of Ibrutinib and 3000 mL of Octanoic acid were taken together in a round bottom flask and stirred for 1 h at 25° C. Methyl tert-butyl ether (1 L) added to the flask and stirred for 5 minutes at 25° C. to get clear solution. The solution was filtered through micron filter to get particle free solution and additional Methyl tert-butyl ether (2 L) was added to the obtained particle free solution and n-Heptane (13.5 L) added to the flask over a period of 20-30 minutes at 25° C. The reaction mass was stirred for 12 h at 25° C. The precipitated material was filtered and washed with n-Heptane (1.5 L) and dried under vacuum suction under further dried in vacuum tray drier.

Yield: 840 g.

### Example 8: Preparation of Crystalline Ibrutinib Form D19

4.0 g of Ibrutinib and 20 mL of Hexanoic acid were taken together and stirred at 25° C. The contents were heated to 40° C. and then cooled to 25° C. 30 mL of n-heptane and 30 mL of methyl tert-butyl ether were added and stirred for 18 hours at 5° C. The contents were filtered at 25° C. and dried in vacuum tray drier at 40° C. for about 6-7 hours to afford the title compound.

Result: Form D19

### Example 9: Preparation of Crystalline Ibrutinib Form D19

20.0 g of Ibrutinib and 30 mL of Hexanoic acid were taken together and stirred at 25° C. The contents were heated to 50° C. and then cooled to 25° C. 200 mL of n-hexane and 10 mL of methyl tert-butyl ether were added and stirred for 16 hours at 5° C. The contents were filtered at 25° C. and dried in vacuum tray drier at 40° C. for 24 hours to afford the title compound.

Result: Form D19

### Example 10: Preparation of Crystalline Ibrutinib Form D19

750 g of Ibrutinib and 3750 mL of Hexanoic acid were taken together in a round bottom flask and stirred for 1 h 30 minutes at 25° C. Methyl tert-butyl ether (3750 mL) added to the flask and stirred for 15 minutes at 25° C. to get clear solution. The solution was filtered through micron filter to get particle free solution and stirred for 20 minutes and n-Heptane (22.5 L) added to the flask over a period of about 2 h at 25° C. The reaction mass was stirred for 22 hrs at 25° C. The precipitated material was filtered and washed with n-Heptane (1.5 L) and dried under vacuum suction and further dried in vacuum tray drier.

Yield: 900 g.

### Example 11: Preparation of Crystalline Ibrutinib Form D20a

2.0 g of Ibrutinib was taken in 5.0 mL of Butyric acid at 25° C. 3 mL of methyl tert-butyl ether and 25 mL of n-Heptane were added to the above contents and stirred for 2-3 hours. The contents were filtered at 25° C. to obtain the title compound.

Result: Form D20a

### Example 12: Preparation of Crystalline Ibrutinib Form D20

Form-D20a was dried in Vacuum tray drier for about 12-72 hours at 25° C. to obtain the title compound.

Result: Form D20

### Example 13: Preparation of Crystalline Ibrutinib Form D21

2.0 g of Ibrutinib and 5 mL of Pentanoic acid were taken together and stirred at 25° C. 25 mL of n-heptane and 3 mL of methyl tert-butyl ether were added and stirred for 3 hours at 25° C. The contents were filtered at 25° C. and dried in vacuum tray drier at 25° C. for 72 hours to afford the title compound.

Result: Form D21

### Example 14: Preparation of Crystalline Ibrutinib Form D22

20.0 g of Ibrutinib and 60 mL of Pentanoic acid were taken together and stirred at 25° C. The contents were cooled to 5° C. and 540 mL of n-heptane and 100 mL of methyl tert-butyl ether were added and stirred for 4 hours at 5° C. The contents were filtered at 25° C. and dried in vacuum tray drier at 25° C. for 42 hours to afford the title compound.

Result: Form D22

### Example 15: Preparation of Crystalline Ibrutinib Form D23

4.0 g of Ibrutinib and 25 mL of Nonanoic acid were taken together and stirred at 25° C. for 50 minutes and filtered the solution to make particle free. 25 mL of methyl tert-butyl ether followed by 150 mL of n-heptane were added and stirred for 28 hours at 25° C. The contents were filtered at 25° C. and dried in vacuum tray drier at 40° C. for 24-48 hours to afford the title compound.

Result: Form D23

### Example 16: Preparation of Crystalline Ibrutinib Form D24

4.0 g of Ibrutinib was dissolved in 25 mL of Heptanoic acid at 40° C. The above clear solution was charged in a 250 mL round bottom flask. Water (100 mL) was added to the above clear solution. Cooled it down to 10° C. and after 24 hours added 100 mL of n-Hexane to it at 25° C. The reaction was maintained for four days. The contents were filtered at 25° C. and dried in vacuum tray drier at 40° C. for 20-24 hours to afford the title compound.

Result: Form D24

### Example 17: Preparation of Crystalline Ibrutinib Form D24

2.5 g of Ibrutinib was dissolved in 6 mL of Heptanoic acid under stirring at 25° C. 6 mL of methyl tert-butyl ether followed by 30 mL of n-pentane were added and stirred for 1 hour at 25° C. The contents were cooled to 5° C. and maintained for 4 days. The contents were filtered at 25° C. to afford the title compound.

Result: Form D24

1. Crystalline Form D18 of Ibrutinib characterized by an X-ray powder diffraction pattern comprising the peak at about 4.23, 8.43 and 12.65±0.20 degrees 2-theta.

2. Crystalline Form D18 of Ibrutinib of claim 1, further characterized by PXRD pattern having additional peaks at about 16.89, 17.33, 18.70, 21.13 and 24.05±0.20 degrees 2-theta.

3. Crystalline Form D18 of Ibrutinib of claim 1, characterized by an X-ray Powder Diffraction Pattern (PXRD) as shown FIG. 5.

4. Crystalline Form D18 of Ibrutinib according to claim 1, wherein said form is an Octanoic acid solvate.

5. A process for preparing crystalline Form D18 of Ibrutinib, comprising the steps of;
a) mixing Ibrutinib and Octanoic acid;
b) mixing an anti-solvent with content of step a); and
c) isolating the Octanoic acid solvate of Ibrutinib.

6. The process of claim 5, wherein the anti-solvent is mixture of methyl tert-butyl ether and n-heptane.

7. Crystalline Form D19 of Ibrutinib characterized by an X-ray powder diffraction pattern comprising the peak at about 4.48, 8.97 and 12.25±0.20 degrees 2-theta.

8. Crystalline Form D19 of Ibrutinib of claim 7, further characterized by PXRD pattern having additional peaks at about 18.05, 19.90, 21.49 and 23.26±0.20 degrees 2-theta.

9. Crystalline Form D19 of Ibrutinib of claim 7, characterized by an X-ray Powder Diffraction Pattern (PXRD) as shown FIG. 8.

10. Crystalline Form D19 of Ibrutinib according to claim 7, wherein said form is a Hexanoic acid solvate.

11. A process for preparing crystalline Form D19 of Ibrutinib of claim 7, comprising the steps of;
a) mixing Ibrutinib and Hexanoic acid;
b) mixing an anti-solvent with content of step a); and
c) isolating the Hexanoic acid solvate of Ibrutinib.

12. The process of claim 11 wherein, the anti-solvent is mixture of methyl tert-butyl ether and n-heptane.

13. A pharmaceutical composition comprising crystalline Form D18 of Ibrutinib of claim 1 and pharmaceutically acceptable excipients.

14. A pharmaceutical composition comprising crystalline Form D19 of Ibrutinib of claim 7 and pharmaceutically acceptable excipients.

15.-26. (canceled)
